(19) United States
(12) Patent Application Publication
(10) Pub. No.: US 2014/0343434 A1
(43) Pub. Date: Nov. 20, 2014

(54) ELECTRICAL ISOLATION OF CATHETER WITH EMBEDDED MEMORY IN IVUS SYSTEMS

(71) Applicant: ACIST Medical Systems, Inc., Eden Prairie, MN (US)

(72) Inventor: Arcadi Elbert, Sunnyvale, CA (US)

(21) Appl. No.: 13/895,067

(22) Filed: May 15, 2013

(51) Int. Cl. A61B 8/00

(52) U.S. Cl. CPC A61B 8/445; A61B 8/56
USPC 600/467

(57) ABSTRACT

An intravenous ultrasound (IVUS) system can include a catheter which can include memory. Because portions of the catheter are in intimate contact with the patient, electrical signals are isolated for safety. The IVUS system or catheter can thus comprise a catheter interface which provides bidirectional isolation circuitry for coupling the catheter to additional components of the IVUS system. Such circuitry allows for memory operation and communication from within the catheter while remaining electrically isolated. Catheter memory can contain information specific to the catheter such as the type of catheter, information regarding catheter components, information regarding catheter use, and other information useful for catheter operation.

### TECHNICAL FIELD

This disclosure relates generally to an intravascular ultrasound (IVUS) system including a removable catheter with embedded memory.

### BACKGROUND

Intravascular ultrasound (IVUS) imaging is a technique that emits sound energy from a transducer at the tip of a small catheter, which is guided into the coronary arteries of the heart or other internal structures in the body. Sound waves that are reflected from vascular tissues are received by the transducer and sent to the system console, where a high-resolution, cross-sectional image is displayed in real time. The IVUS technique provides in-vivo visualization of the vascular structures and lumens, including the coronary artery lumen, coronary artery wall morphology, and devices, such as stents, at or near the surface of the coronary artery wall. IVUS imaging may be used to visualize diseased vessels, including coronary artery disease. An IVUS catheter will, in general, employ at least one high frequency (e.g., 10 MHz-60 MHz, in some preferred embodiments, 40 MHz-60 MHz) ultrasonic transducer that creates pressure waves for visualization. At least one transducer is typically housed within a surrounding sheath or catheter member and mechanically rotated for 360 degree visualization.

In medical applications where an object is inserted into a patient, safety regulations require the object and electrical signals included therein to be electrically isolated from earth ground. To some extent, this protects the patient from possible incidents such as lightning striking the building since the object in the patient is isolated from ground. In part, this has made it difficult to fully communicate bidirectionally with components that also have to meet these regulations since the required isolation complicates bidirectional communication. For example, in the case of a catheter that interfaces with the IVUS system, providing or receiving information beyond the typical RF signals can be difficult.

### SUMMARY

Embodiments discussed in this disclosure provide systems and methods in which an intravascular ultrasound (IVUS) system can communicate bidirectionally with memory stored within a removable catheter. Embodiments of the system can include a removable imaging catheter configured to generate ultrasound image data and having a transducer for emitting and receiving ultrasound signals and memory. In some embodiments of the IVUS system, the catheter has a proximal end and a distal end, and in further embodiments, the catheter memory is located in the proximal end of the catheter. Embodiments can also include an IVUS engine configured to provide operating instructions to the system and construct ultrasound images and a patient interface module (PIM) that includes a catheter interface, is coupled between the catheter and the IVUS engine, and is configured to provide power and instructions to the catheter, receive catheter data from the catheter, and relay catheter data to the IVUS engine. The system can also include electrical isolation circuitry, which is configured to provide both electrical isolation and two-way communication between the PIM and the memory within the removable catheter.

The electrical isolation circuitry allows for memory to be in the catheter while meeting safety requirements and being in full bidirectional communication with other parts of the IVUS system. In some embodiments, the two-way communication contains clock and data signals between the catheter and the PIM, which can be electrically isolated. The electrical isolation circuitry can be contain in the PIM, and in some embodiments contains a transformer and a rectifier. In some embodiments, the rectifier outputs a substantially DC signal and the transformer defines an isolated ground, which can be the reference ground for the isolation circuitry.

Embodiments of the invention include methods for reading stored catheter data from a catheter into an IVUS system. The method can include providing an IVUS catheter with memory that contains catheter data and an IVUS system with a catheter interface, coupling the catheter to the catheter interface and reading stored catheter data from the catheter memory using the IVUS system. In some embodiments, coupling the catheter to the catheter interface results in the catheter interface communicating with the memory in the catheter in such a way so that the catheter is electrically isolated from components of the IVUS system.

In some embodiments, the catheter interface includes isolation circuitry, which can include a bidirectional isolation component. This component can be a hot-swappable component. The stored catheter data can include usage data of the catheter. In some embodiments, the method can include steps of removing the catheter from the catheter interface and coupling a second catheter to the interface. The second catheter can include a second catheter memory, and can be coupled to the interface so that it communicates with the interface and remains electrically isolated from components of the IVUS system. The method can further include reading stored catheter data from the second catheter memory using the IVUS system.

Other embodiments of the invention include methods for attaching an IVUS catheter to an IVUS system. Such methods can include providing an IVUS catheter which includes memory and an IVUS system with a catheter interface and interface circuitry, securing the catheter to the catheter interface, and supplying, from the interface circuitry, at least data, power and ground signals to the catheter memory, such that each of the data, power and ground signals are isolated signals. In some embodiments, the method further includes supplying an isolated clock signal as well. The interface circuitry can include a bidirectional isolation component which provides signals to the catheter interface.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is an illustrative embodiment of an IVUS system.

FIG. 2 is a block diagram of an IVUS system embodiment.

FIG. 3 is an exemplary piece of memory that can be used as the catheter memory.

FIG. 4 is a schematic block diagram of an electrical isolation circuit according to some embodiments of the IVUS system.

FIG. 5 is an exemplary series of signals at various points of the isolation circuitry according to certain embodiments of the IVUS system.

FIG. 6 is a schematic diagram showing an exemplary embodiment of the bidirectional isolation component of the IVUS system.

### DETAILED DESCRIPTION

The following detailed description is exemplary in nature and is not intended to limit the scope, applicability, or configuration of the invention in any way. Rather, the following description provides some practical illustrations for implementing examples of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives.

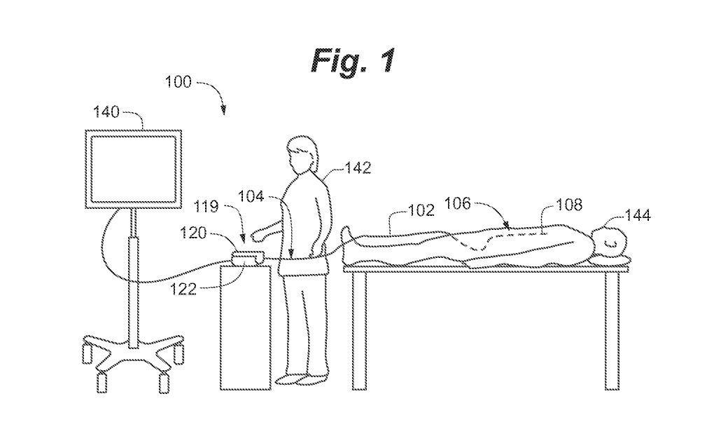

FIG. 1 is an illustrative embodiment of an IVUS system. The IVUS system 100 of FIG. 1 includes a removable catheter 102 having a proximal end 104 and a distal end 106 for inserting into an artery of a patient 144 for imaging. The catheter 102 may be inserted into the patient 144 via the femoral artery, for example. In FIG. 1, broken lines represent portions of the catheter 102 inside the patient's 144 body. According to certain embodiments, the catheter 102 can include a transducer 108 at or near its distal end 106. To perform an imaging function, the transducer 108 can emit ultrasound pulses. The ultrasound pulses can then reflect off the tissue of a patient 144 and can be detected by the transducer 108, which can convert the reflected ultrasound pulses into an electrical signal for image construction. Accordingly, an integrated ultrasound generator may be included in the IVUS system.

The IVUS system 100 of FIG. 1 also includes a translation mechanism. As shown, the translation mechanism 119 includes a patient interface module (PIM) 120 and a linear translation system (LTS) 122. The LTS 122 can be mechanically engaged with the catheter 102. The LTS can be configured to translate the catheter 102 a controlled distance within the patient 144 during a pullback or other translation operation. In this embodiment, the PIM 120 of the translation mechanism 119 also acts as an interface with the catheter 102.

The IVUS system 100 can include a user interface 140 that can receive commands by a system user 142 and/or display IVUS data acquired from the catheter 102 (e.g., as IVUS images). The user interface 140 may include a traditional PC with software configured to communicate with the other components of the IVUS system 100. In some embodiments, the user interface 140 may include a display configured to display system information and/or IVUS signals from the catheter 102 (e.g., as IVUS images). In some embodiments, the user interface 140 can include a touchscreen display, which can act to both receive commands from a system user 142 and display IVUS data from the catheter 102. In some embodiments, the user interface 140 can include an imaging engine configured to construct images from the IVUS data provided by the catheter 102, such as ultrasound signals provided by the transducer 108. In some embodiments, the user interface 140 can include or be in communication with the ultrasound generator.

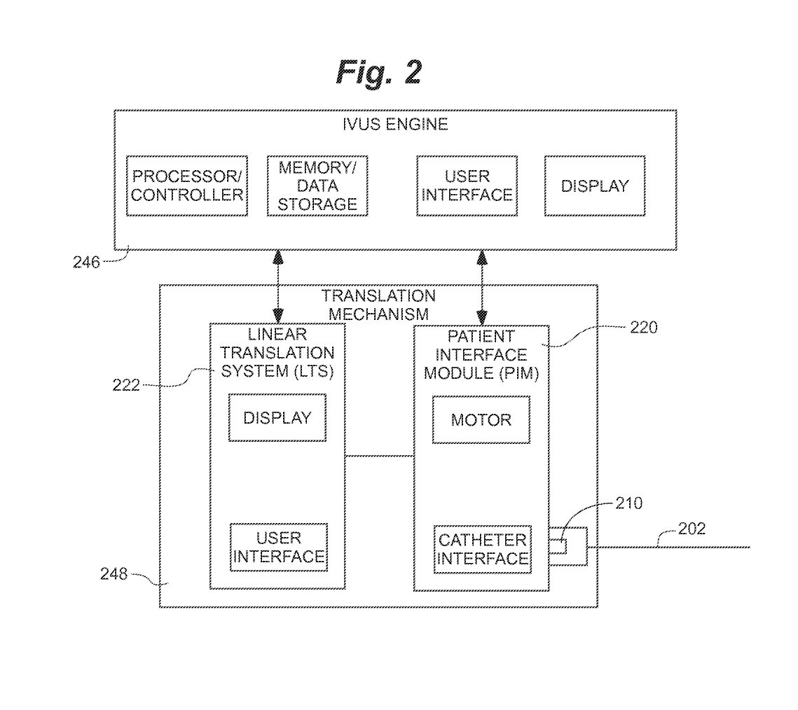

FIG. 2 is a block diagram of an IVUS system embodiment. In some embodiments, the IVUS engine 246 (e.g., an imaging engine) can include a processor/controller, memory/data storage, a user interface, and a display (among other possible components). These components may be integrated into, for example, a touch screen display and/or a computer. The IVUS engine 246 can generally be in communication with a translation mechanism 248, configured to translate the catheter 202 or a portion of the catheter 202. The translation mechanism 248 can, in some embodiments, include its own display and user interface. The translation mechanism 248 and user interface can allow the translation mechanism 248 to be used in a manual mode without requiring operating instructions from the IVUS engine 246. In some embodiments, the translation mechanism 248 can include a motor that can be used to adjust the position of the transducer at the distal end of the catheter 202 rotationally and/or translationally.

In some embodiments, the translation mechanism 248 can include a linear translation system (LTS) 222. The LTS 222 can include the aforementioned display and interface for allowing manual operation of the translation mechanism 248. In some embodiments, the translation mechanism 248 can include a patient interface module (PIM) 220. The PIM 220 can include a catheter interface, which can be attachable to the catheter 202. In some embodiments, the PIM 220 can include the aforementioned motor for adjusting the position of the transducer at the distal end of the catheter 202. According to some embodiments, a translation system 248 can include both a PIM 220 and an LTS 222. In such embodiments, the PIM 220 and the LTS 222 may be fixedly attached to one another. The PIM 220 and LTS 222 may be in communication with one another, and may each individually be in communication with the IVUS engine 246.

In some embodiments of the IVUS system, the transducer on the distal end of the catheter 202 can rotate and/or translate. Rotation of the catheter 202 can be full 360-degree rotation to allow 360-degree imaging of a location such as a patient's artery. In some embodiments, the catheter can be an array catheter, in which rotation need not be necessary for such 360-degree imaging. Translation of the catheter 202 can allow imaging of multiple locations along the artery. Sequential scans can be performed at multiple translation positions to form an aggregate longitudinal image. In some embodiments, the catheter 202 can include a drive cable, which contains an electrical transmission line and is coupled to the transducer. In some embodiments, the catheter 202 can include a sheath defining a lumen within which the transducer and the drive cable are allowed to move freely. Thus, in some embodiments, the transducer can both translate and rotate within the sheath via the drive cable without the sheath moving within the artery. This can be advantageous to avoid excess friction between the catheter and the interior of a patient's artery as the transducer is moved during imaging or other IVUS operations. For example, while moving inside the sheath, the catheter does not drag along vessels which may have plaques prone to rupture.

As mentioned, for some IVUS operations, the transducer can be translated along a length of an artery. To facilitate such a measurement, some embodiments of the IVUS system include a translation mechanism 248. The translation mechanism 248 can engage the catheter 202 and enable the operator of the IVUS system to translate the transducer within the catheter 202 in a specific way. Among various embodiments of the IVUS system, the translation mechanism 248 can translate the catheter 202 a desired distance, at a desired speed, or optionally both. Movement of the transducer can be initiated from the translation mechanism 248 directly and/or from an external controller such as the IVUS engine 246. In the case of an external controller, translation may be performed manually by a user or may be part of an automated process.

In some embodiments of the IVUS system, the translation mechanism 248 can include a PIM 220 and an LTS 222. In some embodiments, the PIM 220 can be configured to attach to the proximal end of the catheter 202. This attachment can include both an electrical and a mechanical attachment. For example, in some embodiments, the PIM 220 can provide the mechanical interface to secure the catheter 202, as well as the mechanical energy to rotate the transducer within the catheter 202. In some embodiments, the PIM 220 can provide the electrical interface that transmits the signal from the integrated ultrasound generator to the catheter 202 and receives the return signal. As such, in some embodiments, the PIM 220 can provide the electromechanical interface between the catheter 202 and the IVUS engine 246.

According to some embodiments, the PIM 220 can be configured to mate to the LTS 222. The LTS 222, while mated with the PIM 220 and catheter 202, can provide longitudinal translation of the transducer. In many embodiments, the longitudinal translation of the transducer can involve pullback of the catheter imaging core at a controlled rate. The LTS 222 can provide calibrated linear translation for acquisition of longitudinal IVUS data (e.g., for imaging). The LTS 222 may feature a display. The display may indicate the linear distance traversed and/or the translation speed. In some embodiments, the display may include controls for starting/stopping translation, setting translation speed, resetting linear distance traversed to zero, switching to manual mode, and so on. In some embodiments, in manual mode, the IVUS system operator can freely move the catheter imaging core forward and backward.

According to some embodiments of the IVUS system, the removable catheter 202 can include catheter memory 210. Thus, if the catheter 202 is removed from the system, the catheter memory 210 can remain with the catheter 202. This way, information that is deemed important to a specific catheter 202 can be kept with that particular catheter 202. In certain embodiments, the catheter memory 210 is located on the proximal end of the catheter 202. The catheter memory can contain information specific to the catheter 202, such as the model of the catheter 202 and information about the particular components within the catheter 202, such as information about the transducer. Such transducer information can include the frequency response of the transducer, its date of assembly, gain, output level, number of times mated to the IVUS system and other transducer-specific information. The memory can also store catheter 202 and/or transducer usage information such as usage time, date, and duration, as well as information regarding the patient in which the catheter 202 was used. Storing such information in the catheter memory 210 guarantees that this information is associated with the correct catheter 202, and that the IVUS engine 246 can detect this information upon catheter 202 engagement or upon the engine 246 requesting such information.

Catheter memory 210 can comprise many types of memory, though preferably the memory 210 is a non-volatile memory such as flash memory or EEPROM (electrically erasable programmable read only memory) so as to retain its information when not powered (i.e. when the catheter 202 is not powered). In order to be functional within the IVUS system, the catheter memory 210 must be in communication with at least one other component of the IVUS system. In some embodiments, the catheter memory 210 is accessed by the PIM 220 via the catheter interface, which can comprise or be connected to interface circuitry. Via the PIM 220, the IVUS engine 246 can gain access to the memory 210 read data stored information or, in some embodiments, write information to be stored.

FIG. 3 is an exemplary piece of memory that can be used as the catheter memory. FIG. 3 shows memory 310 comprising four terminals—power 330, clock 332, data 334, and ground 336. Alternative embodiments may have more or fewer than four terminals for a variety of applications. In an exemplary embodiment, a voltage is provided to memory 310 via the power terminal 330 relative to the ground terminal 336. Data desired to be written to memory is placed on the data terminal 334, and is written to terminal in response to a signal on the clock terminal 332. Data may be read from the data terminal 334 in a similar way.

Since the catheter is inserted into the patient, safety standards require that all electrical components of the catheter be electrically isolated from earth ground. That is, components of the catheter cannot be held at a voltage with an absolute reference to earth ground. This is done to prevent dangerous electrical currents from flowing through the patient to ground. Thus, for memory such as that shown in FIG. 3 to be implemented into the catheter of the IVUS system and to remain electrically isolated from earth ground, each of the power, data, clock and ground terminals must be isolated. Moreover, at least the data terminal needs to be in bidirectional communication with the catheter interface in order for the IVUS engine to read from and write to the memory.

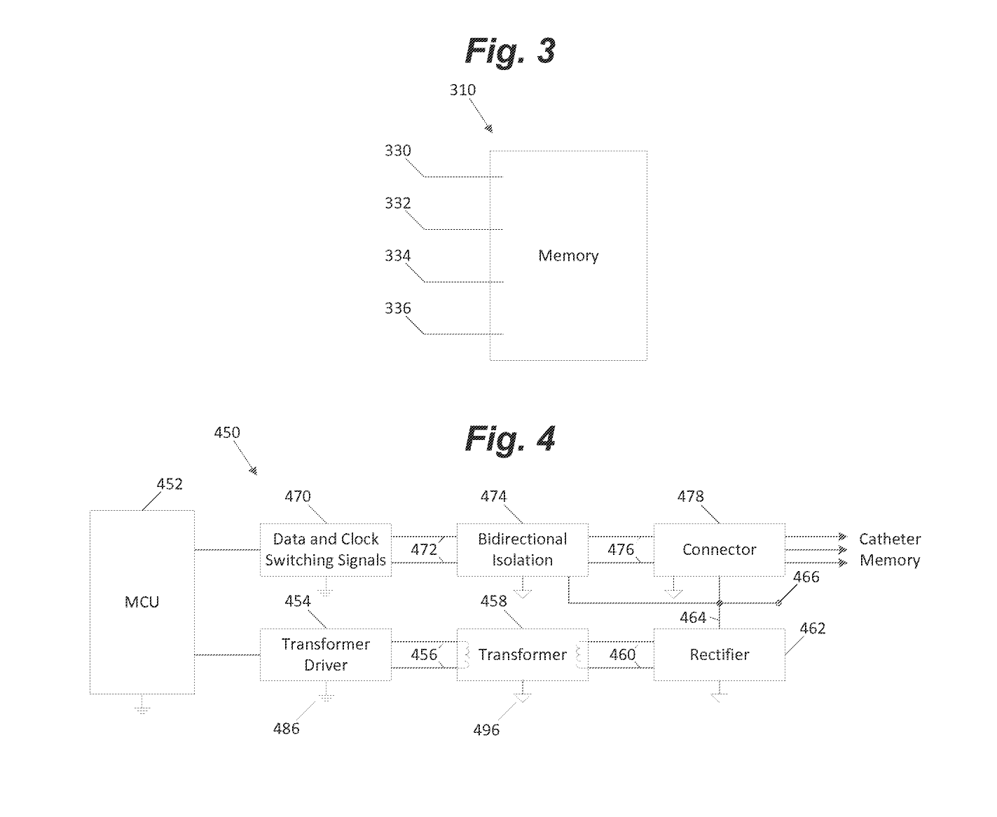

FIG. 4 is a schematic block diagram of an electrical isolation circuit according to some embodiments of the IVUS system. FIG. 4 shows an isolation circuit 450 ultimately showing communication between a microcontroller 452 and a connector 478 which may connect to the catheter, while providing isolation between the connector and earth ground. Referring first to the bottom half of the circuit 450, the microcontroller 452 is coupled to a transformer driver 454, which sends a primary signal 456 through a transformer 458. In some embodiments, the primary signal 456 through the transformer is a square wave or a substantially square wave, though other waveforms are contemplated. The microcontroller 452 and transformer driver 454 are coupled to and provide signals with reference to earth ground 486. The transformer 458 receives the primary signal 456 from the transformer driver, which induces a secondary signal 460 in the transformer which is floating. That is, the secondary signal 460 is with reference to an isolated ground 496, which is not associated with earth ground 486. In an exemplary embodiment, if a square wave with reference to earth ground 486 was applied to the transformer 458, a secondary square wave with reference to an isolated ground 496 is produced from the transformer 458. It should be noted that in FIG. 4, reference numerals 486 and 496 refer to symbols representing earth ground and isolated ground, respectively. Unless otherwise noted, these symbols should be taken to represent the associated ground, even when a reference numeral is absent.

The transformer 458 in the embodiment of FIG. 4 outputs the isolated secondary signal 460 to a rectifier 462, which in turn outputs a rectified signal 464. As shown, rectifier 462 is referenced to the isolated ground 496. In some embodiments, rectifier 462 is a full-wave rectifier. Thus, in some embodiments, the microcontroller 452 tells the transformer driver 454 to output a square wave to a transformer 458, which outputs an isolated square wave to a full-wave rectifier 462. If the incident isolated square wave is centered about the isolated ground 496, the resulting output 464 from the rectifier 462 will be substantially an isolated DC signal. That is, the rectified signal 464 from the rectifier 462 will be substantially constant with respect to isolated ground 496. It is considered a substantially isolated DC signal because in some embodiments, it is possible that the signal might not be exactly constant at the transition points in the incident square wave. Additionally, in some embodiments, the secondary signal 460 from the transformer 458 may not be a perfect square wave, but rather may have some variation while remaining substantially a square wave. In such a case, the rectified signal 464 may reflect this variation, rendering it a substantially DC signal. The true rectified signal 464 can be measured, for example, at node 466.

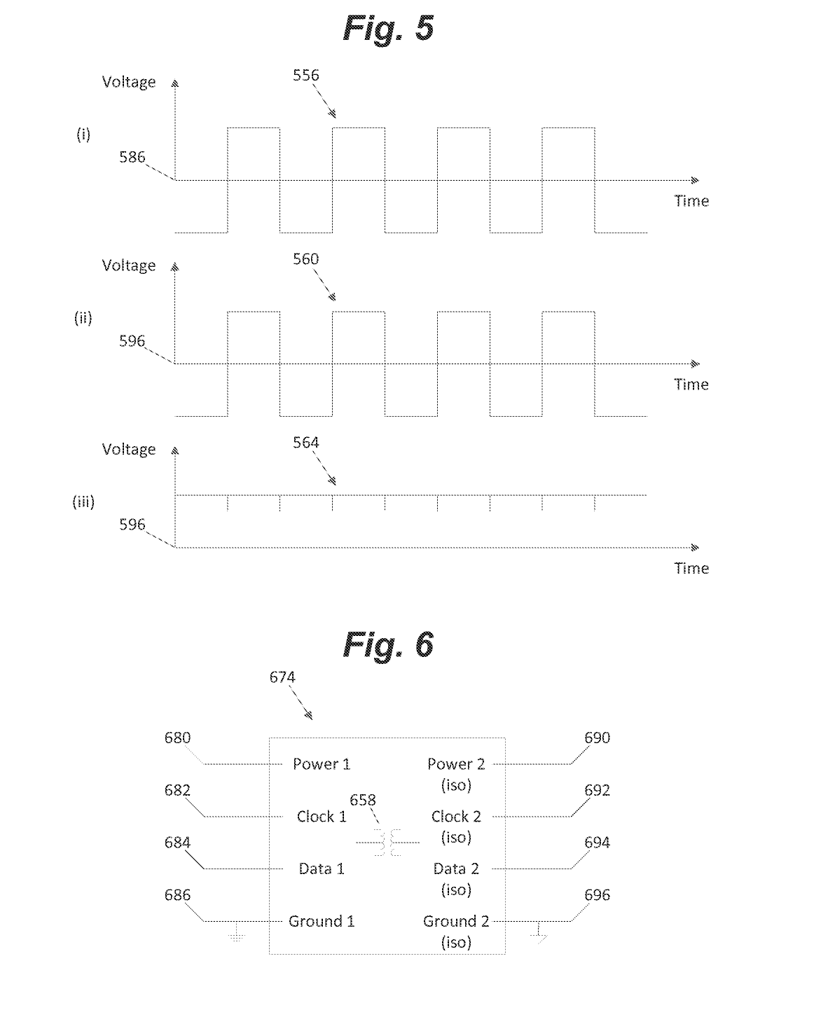

FIG. 5 is an exemplary series of signals at various points of the isolation circuitry according to certain embodiments of the IVUS system. Signal (i) in FIG. 5 shows an exemplary primary signal 556 outputted from a transformer driver 454 such as the one in FIG. 4. The primary signal 556 is a square wave centered about earth ground 586. Signal (ii) in FIG. 5 shows an exemplary secondary signal 560, as may be outputted from the transformer 458 when the primary signal 556 of (i) is inputted from the transformer driver 454. Secondary signal 560 is also a square wave, only it is centered around the isolated ground 596 as opposed to earth ground 586. It should be noted, though, that in some embodiments the transformer may not output a perfect square wave. Finally, signal (iii) of FIG. 5 shows an exemplary rectified signal 564, as may be outputted from the rectifier 462 when the secondary signal 560 of (ii) is inputted from the transformer 458. The rectified signal 564 is substantially a DC signal referenced to the isolated ground 596, though it may contain some irregularity at the transition points of the secondary signal 560. It should be noted that the voltage and time scales of FIG. 5 are arbitrary, but in some embodiments, each signal could be plotted on the same scale.

Referring now to the top half of FIG. 4, the microcontroller 452 controls data and clock switching signals 470 intended to send data and clock signals 472 to the catheter. These signals 472, however, are referenced to earth ground 486. The signals 472 are sent to a bidirectional isolation component 474, which has reference to both earth ground 486 and the isolated ground 496 from the transformer 458. Generally, the bidirectional isolation component 474 can compare the incident signals 472 relative to earth ground 486, and output isolated signals 476 relative to the isolated ground 496.

Accordingly, in some embodiments, such as that of FIG. 4, isolated data, clock, power and ground signals scan be sent from the combination of the bidirectional isolation component 474 and the rectifier 462 to a connector 478 configured to engage with the catheter. Memory in the catheter can therefore receive one or more signals necessary for operation while being completely isolated from earth ground, therefore providing a safe environment for the patient.

In some embodiments of the IVUS system, the bidirectional isolation component 474 comprises a bidirectional, hot-swappable isolating component which enables a fully replaceable, bidirectional electrical component to be in full electrical communication with an electrical device such as the PIM or the IVUS engine while remaining electrically isolated from it. Generally a hot-swappable device refers to one that can be removed during operation, thus in some embodiments potentially allowing for catheters to be changed ‘on the fly’ without shutting down power. Further, in some embodiments, the bidirectional isolation component 474 can provide 5000 volts isolation, meaning that up to a 5000 volt potential can be placed across the bidirectional isolation component 474 without it breaking down and eliminating the isolation. In various embodiments, various isolation voltage ratings can be used depending on the requirements of operation.

FIG. 6 is a schematic diagram showing an exemplary embodiment of the bidirectional isolation component of the IVUS system. FIG. 6 shows a bidirectional isolation component 674 with first power 680, first clock 682, first data 684, and first ground 686 inputs. In this embodiment, first power 680, first clock 682, and first data 684 are all signals with reference to the first ground 686, which is earth ground. These inputs can receive signals from earth-grounded components from the IVUS system such as a PIM or an IVUS engine, for example.

The bidirectional isolation component 674 can determine the signals 682 and 684 with respect to the first ground 686 and power 680, and output corresponding signals 692 and 694 with respect to the second ground 696 and power 690. Thus, if the first ground 686 is earth ground and the second ground 696 is isolated ground, the same relative voltages can be applied relative to the isolated ground via the component's output as were applied at its input relative to earth ground.

The bidirectional properties of the bidirectional isolation component 674 allow a similar procedure to occur in the other direction. In this case, isolated signals, for example from the catheter memory, can be applied to the second power 690, second clock 692, second data 694 and second ground 696, and signals such as first clock 682, and first data 684 can be output with respect to the first ground 686, which can be earth ground. This configuration allows catheter memory to communicate fully with the PIM and/or the IVUS engine while remaining electrically isolated therefrom.

The bidirectional isolation component 674 of FIG. 6 is shown having a transformer symbol 658 between first and second components. While in some embodiments of the invention the bidirectional isolation component 674 may contain at least one transformer, the symbol 658 are included in FIG. 6 merely to indicate that the signals on either side of the transformer symbol are isolated from one another. Moreover, the bidirectional isolation component 674 can be a hot-swappable device, which in some embodiments allows multiple catheters to be interchanged during operation of the IVUS system without requiring shutdown.

Thus, in some embodiments of the IVUS system, removable catheters which include memory can be coupled to the IVUS system and communicate with the IVUS engine while remaining electrically isolated from the system and earth ground. Such embodiments allow for catheter-specific information to be stored in memory in an individual catheter and communicated to the IVUS system while meeting electrical isolation safety standards for devices such as catheters. Various examples have been described. These and other examples are within the scope of the following claims.

1. An IVUS system, comprising:
an imaging catheter for inserting into the vasculature of a patient and configured to generate ultrasound image data, the imaging catheter comprising memory and a transducer for emitting and receiving ultrasound signals;
an IVUS engine configured to provide operating instructions to the system and receive ultrasound image data and construct ultrasound images;
a patient interface module (PIM) (i) including a catheter interface, (ii) being removably connectable to the imaging catheter via the catheter interface; and (iii) being configured to provide power and instructions to the imaging catheter, receive catheter data from the imaging catheter, and relay catheter data to the IVUS engine; and
electrical isolation circuitry, configured to provide both electrical isolation and two-way communication between the PIM and the memory of the imaging catheter.

2. The IVUS system of claim 1, wherein the imaging catheter comprises a proximal end and a distal end, and the catheter memory is located in the proximal end of the imaging catheter.

3. The IVUS system of claim 1, wherein the two-way communication between the PIM and the memory of the imaging catheter comprises clock and data signals.

4. The IVUS system of claim 3, wherein the clock and data signals are electrically isolated from earth ground.

5. The IVUS system of claim 1, wherein the electrical isolation circuitry is contained in the PIM.

6. The IVUS system of claim 1, wherein the electrical isolation circuitry comprises a transformer and a rectifier.

7. The IVUS system of claim 6, wherein the rectifier outputs an electrically isolated, substantially DC signal.

8. The IVUS system of claim 6, wherein the transformer defines an isolated ground.

9. The IVUS system of claim 8, wherein the isolated ground defined by the transformer provides the reference ground for the electrical isolation circuitry.

10. The IVUS system of claim 1, wherein electrical isolation circuitry provides isolated power to the memory in the imaging catheter.

11. The IVUS system of claim 1, wherein the electrical isolation circuitry provides up to 5000 volts isolation.

12. The IVUS system of claim 1, wherein the electrical isolation circuitry comprises a hot-swappable, bidirectional component.

13. A method for reading stored catheter data from a catheter into an IVUS system comprising:
providing an IVUS catheter with internal catheter memory, the memory comprising catheter data;
providing an IVUS system with a catheter interface;
removably coupling the IVUS catheter to the catheter interface, such that the catheter interface communicates with the memory in the IVUS catheter via at least one electrical signal and wherein the at least one electrical signal is electrically isolated from earth ground;
reading stored catheter data from the catheter memory using the IVUS system.

14. The method of claim 13, wherein the stored catheter data comprises usage data of the catheter.

15. The method of claim 13, wherein the catheter interface comprises isolation circuitry.

16. The method of claim 15, wherein the isolation circuitry comprises a bidirectional isolation component.

17. The method of claim 16, wherein the bidirectional isolation component is a hot-swappable component.

18. The method of claim 17, further comprising the steps of (i) removing the IVUS catheter from the catheter interface; (ii) coupling a second IVUS catheter comprising a second catheter memory to the catheter interface, such that the catheter interface communicates with the second catheter memory via at least a second electrical signal and wherein the second electrical signal is electrically isolated from earth ground; and (iii) reading stored catheter data from the second catheter memory using the IVUS system.

19. The method of claim 13, wherein the portion of the IVUS system that reads the stored catheter data from the catheter memory comprises an IVUS engine.

20. The method of claim 19, wherein the IVUS engine communicates with the IVUS catheter via a patient interface module (PIM).

21. A method for attaching an IVUS catheter to an IVUS system comprising:
providing an IVUS catheter comprising catheter memory;
providing an IVUS system comprising a catheter interface including interface circuitry and configured to mate with the IVUS catheter;
removably securing the IVUS catheter to the catheter interface;
supplying, from the interface circuitry, at least data, power and ground signals to the catheter memory; wherein
the data, power and ground signals; each signal being electrically isolated from earth ground.

22. The method of claim 21, further comprising supplying, from the interface circuitry, an isolated clock signal.

23. The method of claim 21, wherein the interface circuitry comprises a bidirectional isolation component which provides isolated data, power and ground signals to the catheter interface.

24. The method of, claim 23, wherein the bidirectional isolation component comprises a hot-swappable element.

25. The method of claim 21, the isolated power signal is a substantially DC signal.
